SEQUENCE LISTING (23 sequences)

SEQ ID NO: 1 - protein, 572 aa, Campylobacter jejuni
MGFRINTNVAALNAKANADLNSKSLDASLSRLSSGLRINSAADDASGMAIADSLRSQANTLGQAISNGNDALGILQTADKAMDEQLKILDTIKTKATQAAQDGQSLKTRTMLQADINRLMEELDNIANTTSFNGKQLLSGNFINQEFQIGASSNQTVKATIGATQSSKIGLTRFETGGRISTSGEVQFTLKNYNGIDDFQFQKVVISTSVGTGLGALADEINKNADKTGVRATFTVETRGIAAVRAGATSDTFAINGVKIGKVDYKDGDANGALVAAINSVKDTTGVEASIDANGQLLLTSREGRGIKIDGNIGGGAFINADMKENYGRLSLVKNDGKDILISGSNLSSAGFGATQFISQASVSLRESKGQIDANIADAMGFGSANKGVVLGGYSSVSAYMSSAGSGFSSGSGYSVGSGKNYSTGFANAIAISAASQLSTVYNVSAGSGFSSGSTLSQFATMKTTAFGVKDETAGVTTLKGAMAVMDIAETAITNLDQIRADIGSVQNQVTSTINNITVTQVNVKAAESQIRDVDFAAESANYSKANILAQSGSYAMAQANSVQQNVLRLLQ

SEQ ID NO: 2 - DNA, 1719 bp, Campylobacter jejuni
atgggatttcgtattaacaccaatgttgcagctttaaatgcaaaagcaaacgctgatttaaatagtaaaagtttagatgcttctttaagcagacttagttcaggtcttagaatcaactccgcagcagatgatgcttcagggatggcgatagcagatagtttaagatctcaagctaatactttaggtcaagctatatctaatggtaatgatgctttaggtatcttacaaactgctgataaggctatggatgagcaacttaaaatcttagatacaatcaaaactaaggcaactcaagcggctcaagatggacaaagtttaaaaacaagaaccatgcttcaagcagatatcaaccgtttaatggaagaacttgacaatattgcaaatactacttcatttaacggtaaacaacttttaagtgggaattttatcaatcaagaatttcaaatcggtgcaagttcaaatcaaactgtaaaagctactataggagcaactcaatcttctaagataggtttaacacgctttgaaacaggaggaagaatttcaactagtggcgaagtacaatttactcttaaaaattacaatggtatagatgattttcagtttcaaaaagttgtgatttcaacttcagttggaacaggacttggagctttagcagatgagatcaataaaaatgctgataaaacaggtgttagagctacttttacagtagaaactagaggtatagctgcagttagagcaggagctacttcagatacttttgctatcaatggggtaaaaatcggtaaagtagattacaaagatggtgatgctaatggagccttagttgctgcaatcaattcggttaaagataccactggagttgaagcttcgatcgatgctaatggacaacttttacttacttcaagagaaggtagagggattaaaatcgatggtaatataggtggaggtgcctttatcaatgctgatatgaaagaaaactatggccgcttgtctttagttaaaaatgatggtaaagatattttaatcagcggtagcaatctttcttctgcaggttttggtgcaactcaatttatctctcaagcttctgtttctttaagagagtcaaaaggacaaattgatgctaatatcgctgatgctatgggatttggttctgcaaacaaaggagttgtgttaggtggttattcttctgttagtgcctatatgagtagcgcaggaagtggattttcttcaggttcaggttattctgtaggtagcggtaaaaattattccacaggttttgcaaacgctatagctatttcagctgcttcgcaactttctacggtatataatgtttctgcaggctcaggtttttcaagtggttcaacactttctcagtttgcaactatgaaaacaactgcttttggagtaaaagatgaaacagcaggtgttaccacacttaaaggcgctatggctgtgatggatatagctgaaacagctataacaaatcttgatcaaatcagagccgacattggttcggtacaaaatcaagttacatcaactataaacaacatcaccgtaactcaagtaaacgttaaagcagcagaatcgcaaatccgtgatgtagactttgcagccgagagtgcaaactactctaaagcaaatatcttagctcaaagcggctcttatgccatggcacaggctaattctgttcaacaaaatgttttaagattactacagtag

SEQ ID NO: 3 - protein, 572 aa, Campylobacter jejuni
MGFRINTNIGALNAHANSVVNARELDKSLSRLSSGLRINSAADDASGMAIADSLRSQAATLGQAINNGNDAIGILQTADKAMDEQLKILDTIKTKATQAAQDGQSLKTRTMLQADINRLMEELDNIANTTSFNGKQLLSGNFINQEFQIGASSNQTIKATIGATQSSKIGLTRFETGGRISSSGEVQFTLKNYNGIDDFQFQKVVISTSVGTGLGALAEEINKSADKTGVRATFTVETRGIAAVRAGTTSDTFAINGVTIGQVAYEDGDGNGALVAAINSVKDTTGVEASIDANGQLLLTSREGRGIKIDGNIGGGAFINADMKENYGRLSLVKNDGKDILISGSNLSSAGFGATQFISQASVSLRESKGRFDANIADAMGFGSANKGVVLGGYSSVSAYMSSAGSGFSSGSGYSVGSGKNYSTGFANAIAISAASQLSTVYNVSAGSGFSSGSTLSQFATMKTTAFGVKDETAGVTTLKGAMAVMDIAETATTNLDQIRADIGSVQNQLQVTINNITVTQVNVKAAESTIRDVDFAAESANFSKYNILAQSGSYAMSQANAVQQNVLKLLQ

SEQ ID NO: 4 - protein, 273 aa, Campylobacter jejuni
MKKFSLVAATLIAGVVLNVNAATVATVNGKSISDTEVSEFFAPMLRGQDFKTLPDNQKKALIQQYIMQDLILQDAKKQNLEKDPLYTKELDRAKDAILVNVYQEKILNTIKIDAAKVKAFYDQNKDKYVKPARVQAKHILVATEKEAKDIINELKGLKGKELDAKFSELAKEKSIDPGSKNQGGELGWFDQSTMVKPFTDAAFALKNGTITTTPVKTNFGYHVILKENSQAKGQIKFDEVKQGIENGLKFEEFKKVINQKGQDLLNSAKVEYK

SEQ ID NO: 5 - protein, 250 aa, Campylobacter jejuni
MKKIITLFGACALAFSMANADVNLYGPGGPHTALKDIANKYSEKTGVKVNVNFGPQATWFEKAKKDADILFGASDQSALAIASDFGKDFNVSKIKPLYFREAIILTQKGNPLKIKGLKDLANKKVRIVVPEGAGKSNTSGTGVWEDMIGRTQDIKTIQNFRNNIVAFVPNSGSARKLFAQDQADAWITWIDWSKSNPDIGTAVAIEKDLVVYRTFNVIAKEGASKETQDFIAYLSSKEAKEIFKKYGWRE

SEQ ID NO: 6 - protein, 245 aa, Campylobacter jejuni
MKKILILSLAASFLNAEILVYGPGGPAPVLKELALKFEEKTKEKVIVTAGPTPAWVDKAKENADLIFSGNTSMMDDFAKKIPSLSLENLSVLNVRPSGIIVRPNNPKNIKNFEDILKDGINVMVVDGAGQVGLYEDMALKSAKRENLVKLRKNIKIYAKNSKAAVDEWNNNPNIDALIIWSHWAKALGDDKALFIKDKNAVIYRAAEIAPTKKGLENKKALEFVDFIKSQEAQKVWKKYTWKEVK

SEQ ID NO: 7 - protein, 259 aa, Campylobacter jejuni
MVFRKSLLKLAVFALGACVAFSNANAAEGKLESIKSKGQLIVGVKNDVPHYALLDQATGEIKGFEVDVAKLLAKSILGDDKKIKLVAVNAKTRGPLLDNGSVDAVIATFTITPERKRIYNFSEPYYQDAIGLLVLKEKKYKSLADMKGANIGVAQAATTKKAIGEAAKKIGIDVKFSEFPDYPSIKAALDAKRVDAFSVDKSILLGYVDDKSEILPDSFEPQSYGIVTKKDDPAFAKYVDDFVKEHKNEIDALAKKWGL

SEQ ID NO: 8 - protein, 319 aa, Campylobacter jejuni
MKKIFLCLGLASVLFGADNNVKFEITPTLNYNYFEGNLDMDNRYAPGIRLGYHFDDFWLDQLEFGLEHYSDVKYTNTNKTTDITRTYLSAIKGIDVGEKFYFYGLAGGGYEDFSNAAYDNKSGGFGHYGAGVKFRLSDSLALRLETRDQINFNHANHNWVSTLGISFGFGGKKEKAVEEVADTRATPQAKCPVEPREGALLDENGCEKTISLEGHFGFDKTTINPTFQEKIKEIAKVLDENERYDTILEGHTDNIGSRAYNQKLSERRAKSVANELEKYGVEKSRIKTVGYGQDNPRSSNDTKEGRADNRRVDAKFILR

SEQ ID NO: 9 - protein, 367 aa, Campylobacter jejuni
MKLFQKNTILALGVVLLLAACSKEEAPKIQMPPQPVTTMSAKSEDLPLSFTYPAKLVSDYDVIIKPQVSGVIVNKLFKAGDKVKKGQTLFIIEQDKFKASVDSAYGQALMAKATFENASKDFNRSKALFSKSAISQKEYDSSLATFNNSKASLASARAQLANARIDLDHTEIKAPFDGTIGDALVNIGDYVSASTTELVRVTNLNPIYADFFISDTDKLNLVRNTQSGKWDLDSIHANLNLNGETVQGKLYFIDSVIDANSGTVKAKAVFDNNNSTLLPGAFATITSEGFIQKNGFKVPQIGVKQDQNDVYVLLVKNGKVEKSSVHISYQNNEYAIIDKGLQNGDKIILDNFKKIQVGSEVKEIGAQ

SEQ ID NO: 10 - protein, 1040 aa, Campylobacter jejuni
MFSKFFIERPVFASVVAIIISLAGAIGLTNLPIEQYPSLTPPTVKVSATYTGADAQTIASTVASPIEDAINGADNMIYMDSTSSSSGTMSLTVYFDIGTDPDQATIDVNNRISAATAKMPDAVKKLGVTVRKTSSTTLAAISMYSSDGSMSAVDVYNYITLNVLDELKRVPGVGDANAIGNRNYSLRIWLKPDLLNKFGITATDVISAVNDQNAQYATGKIGEEPVTQKSPYVYSITMQGRLQNPSEFENIILRTNNDGSFLRLKDVADVEIGSQQYSSQGRLNGNDAVPIMINLQSGANALHTAELVQAKMQELSKNFPKGLTYKIPYDTTKFVIESIKEVVKTFVEALILVIIVMYMFLKNFRATLIPMIAVPVSLLGTFAGLYVLGFSINLLTLFALILAIGIVVDDAIIVVENIDRILHENEQISVKDAAIQAMQEVSSPVISIVLVLCAVFVPVSFISGFVGEIQRQFALTLAISVTISGFVALTLTPSLCALFLRRNEGEPFKFVKKFNDFFDWSTSVFSAGVAYILKRTIRFVLIFCIMLGAIFYLYKAVPNSLVPEEDQGLMISIINLPSASALHRTISEVDHISQEVLKTNGVKDAMAMIGFDLFTSSLKENAAAMFIGLQDWKDRNVSADQIIAELNKKFAFDRNASSVFIGLPPIPGLSITGGFEMYVQNKSGKSYDEIQKDVNKLVAAANQRKELSRVRTTLDTTFPQYKLIIDRDKLKHYNLNMQDVFNTMNATIGTYYVNDFSMLGKNFQVNIRAKGDFRNTQDALKNIFVRSNDGKMIPLDSFLTLQRSSGPDDVKRFNLFPAAQVQGQPAPGYTSGQAIEAIAQVAKETLGDDYSIAWSGSAYQEVSSKGTASYAFALGMIFVFLILAAQYERWLIPLAVVTAVPFAVFGSFLLVYLRGFSNDIYFQTGLLLLIGLSAKNAILIVEFAMEERFKKGKGVFEAAVAAAKLRFRPIIMTSLAFTFGVLPMIFATGAGSASRHSLGTGLIGGMIAASTLAIFFVPLFFYLLENFNEWLDKKRGKVHE

SEQ ID NO: 11 - protein, 492 aa, Campylobacter jejuni
MNKIISISAIASFTLLISACSLSPNLNIPEANYSIDNKLGALSWEKETNSSITKNWWKDFDDENLNKVVDLALKNNNDLKLAFIHMEQAAAQLGIDFSSLLPKFDGSASGSRAKTAINAPSNRTGEVSYGNDFKMGLNLSYEIDLWGKYRDTYRASKSGFKASEYDYEAARLSVISNTVQTYFNLVNAYENENALKEAYESAKEIYRINDEKFQVGAVGEYELAQARANLESMALQYNEAKLNKENYLKALKILTSNDLNDILYKNQSYQVFNLKEFDIPTGISSTILLQRPDIGSSLEKLTQQNYLVGVARTAFLPSLSLTGLLGFESGDLDTLVKGGSKTWNIGGNFTLPIFHWGEIYQNVNLAKLNKDEAFVNYQNTLITAFGEIRYALVARKTIRLQYDNAQASEQSYKRIYEIAKERYDIGEMSLQDYLEARQNWLNAAVAFNNIKYSYANSIVDVIKAFGGGFEQSEDTSKNIKEESKNLDMSFRE

SEQ ID NO: 12 - protein, 545 aa, Campylobacter jejuni
MAKEIIFSDEARNKLYEGVKKLNDAVKVTMGPRGRNVLIQKSFGAPSITKDGVSVAKEVELKDSLENMGASLVREVASKTADQAGDGTTTATVLAHAIFKEGLRNITAGANPIEVKRGMDKACEAIVAELKKLSREVKDKKEIAQVATISANSDEKIGNLIADAMEKVGKDGVITVEEAKSINDELNVVEGMQFDRGYLSPYFITNAEKMTVELSSPYILLFDKKITNLKDLLPVLEQIQKTGKPLLIIAEDIEGEALATLVVNKLRGVLNISAVKAPGFGDRRKAMLEDIAILTGGEVISEELGRTLESATIQDLGQASSVIIDKDNTTIVNGAGEKANIDARVNQIKAQIAETTSDYDREKLQERLAKLSGGVAVIKVGAATETEMKEKKDRVDDALSATKAAVEEGIVIGGGAALIKAKAKIKLDLQGDEAIGAAIVERALRAPLRQIAENAGFDAGVVVNSVENAKDENTGFDAAKGEYVNMLESGIIDPVKVERVALLNAVSVASMLLTTEATISEIKEDKPTMPDMSGMGGMGGMGGMM

SEQ ID NO: 13 - protein, 86 aa, Campylobacter jejuni
MNFQPLGKRVLVKRVEETKTTASGIIIPDNAKEKPLMGEVVAVSKEITDIANGDKIVFAKYGGTEIKLDNNEYLVLNLDDILGILK

SEQ ID NO: 14 - protein, 190 aa, Campylobacter jejuni
MKKVLLSSLVAVSLLSTGLFAKEYTLDKAHTDVGFKIKHLQISNVKGNFKDYSAVIDFDPASAEFKKLDVTIKIASVNTENQTRDNHLQQDDFFKAKKYPDMTFTMKKYEKIDNEKGKMTGTLTIAGVSKDIVLDAEIGGVAKGKDGKEKIGFSLNGKIKRSDFKFATSTSTITLSDDINLNIEVEANEK

SEQ ID NO: 15 - protein, 399 aa, Campylobacter jejuni
MAKEKFSRNKPHVNIGTIGHVDHGKTTLTAAISAVLSRRGLAELKDYDNIDNAPEEKERGITIATSHIEYETDNRHYAHVDCPGHADYVKNMITGAAQMDGAILVVSAADGPMPQTREHILLSRQVGVPYIVVFMNKADMVDDAELLELVEMEIRELLSSYDFPGDDTPIISGSALKALEEAKAGQDGEWSAKIMDLMAAVDSYIPTPTRDTEKDFLMPIEDVFSISGRGTVVTGRIEKGVVKVGDTIEIVGIKDTQTTTVTGVEMFRKEMDQGEAGDNVGVLLRGTKKEEVIRGMVLAKPKSITPHTDFEAEVYILNKDEGGRHTPFFNNYRPQFYVRTTDVTGSIKLADGVEMVMPGENVRITVSLIAPVALEEGTRFAIREGGKTVGSGVVSKIIK

SEQ ID NO: 16 - protein, 104 aa, Campylobacter jejuni
MGKYIELTSDNFAQAKEGVALVDFWAPWCGPCRMLAPVIDELSNDFDGKAKICKVNTDEQGDLAAEFGVRSIPTLIFFKNGEVVDQLVGAQSKQAISDKLNSLL

SEQ ID NO: 17 - protein, 424 aa, Campylobacter jejuni
MKLVKLSLVAALAAGAFSAANATPLEEAIKDVDVSGVLRYRYDTGNFDKNFVNNSNLNNSKQDHKYRAQVNFSAAIADNFKAFVQFDYNAADGGYGANGIKNDQKGLFVRQLYLTYTNEDVATSVIAGKQQLNLIWTDNAIDGLVGTGVKVVNNSIDGLTLAAFAVDSFMAAEQGADLLEHSNISTTSNQAPFKVDSVGNLYGAAAVGSYDLAGGQFNPQLWLAYWDQVAFFYAVDAAYSTTIFDGINWTLEGAYLGNSLDSELDDKTHANGNLFALKGSIEVNGWDASLGGLYYGDKEKASTVVIEDQGNLGSLLAGEEIFYTTGSRLNGDTGRNIFGYVTGGYTFNETVRVGADFVYGGTKTEAANHLGGGKKLEAVARVDYKYSPKLNFSAFYSYVNLDQGVNTNESADHSTVRLQALYKF

SEQ ID NO: 18 - protein, 610 aa, Campylobacter jejuni
MNNFKEIAKLVRKYKERNNALYEFLDKEDVGEYFRSLISLSELKQDKTTMLAILRRLIDLKEENLVQEWKKNNFKEDKIIELKHKFYEEVRKFYEKEHQNLINEIKEKKLLNNFYQSLIQGVHNIGLIMNIFEISWTKEIIEKNNKILSTQFPNLDDAMEFLRKNHLYQKTPEGEICERSYGVLVRIGNLWKFVPYARFFENEILKLEFAFENMIDQLKIFASSEEEKAYIEYFEKLKLAFCEKDEDRVIKAWQEAEFAWMKVKSPLQVGHPLEYYEDNYTHAVALEWDIRIEDENDFDVLKFGNEIKESFEHVYKNIGLEDCELEKEVLSNIEKTQLYICTPMIFYGAELKGLFSAQVVPNDEFVSSKAGKKIFAFINFVYENAKTKPFMKISSEVFDKEFLDFGRNILFYQEKIWKRVYEVSTIGHEFGHIFFIANDTEKTMNQSGFFKNIEEYKATTGGLINFFYHEQDDLIMPVFHELIKRAIGLISWQRVDEVRPYYTEGLIHLSLLFESEVLIFENNNLKINFDLGYYEKFKELTLKNYHELAKHYALRLDAKEFLSRFCEIEDNIFLPIMPKCKEFVKFYYDLYEKIGNEIDNSGEFERYKKK

SEQ ID NO: 19 - protein, 329 aa, Campylobacter jejuni
MRKIALFLSLCVFIWASDLQQALEYEKQGDYKKAMEIYKKLALKNSSVLISQEQNNSSQATQTQNSITIKKEEKQDFSRLALANYLGENESFNPLGISSYKMNYFLPFAYSFNSLGVNNNKSEAKFQLSVKKRLFENLLGLDEKYYIAYTQTSWWQIYEHSSPFRETNYQPEFFIDLPLYLKDYEFFNNLRVGILHESNGKGDENLQSRSWNRIYVSTAILYNKFLFVPRLWYRIPENKKDDDNPAILHYMGNFDVNLAYLGDDYFINLMLRNNLKFHNNKGAIQVDLGYDIFNNGIYWYLQYFNGYGESLIDYNKHLQRLSTGFLISY

SEQ ID NO: 20 - protein, 198 aa, Campylobacter jejuni
MAFKNLKELSFEKSNYIKPKRFAYESNGRLCTWDFIESKDSVSVLLYHKELESFIFVRQFRIPLWYHQMHDKDYVKDDDMGYTIELCSGLVDKKLSLEEIAKEECIEELGYAPKNLEKIGDFYTGFGSGVSKQSFYFVEVDEKDKISSGGGVDDEEIEAVYVKVQDFEKKCKNMIRTPLLDFAYMWFLKEKWGKLKII

SEQ ID NO: 21 - protein, 556 aa, Campylobacter jejuni
  misc_feature (113)..(113): Xaa can be any naturally occurring amino acid
MRYLAIFAISITLSFGAANPPIQDRTGTGLVLSSHELANKIGKEVLDKGGNAIDAAVAVGYALAVVHPAAGNIGGGGFAVIHLANGENTTLDFREMAPLKASRNMYLDSKGEXIKDASTIGYLAAGVPGTVKGMSAMLDRYGTMKLKDLMAPAIKLAEKGYLINDPQEQTMLEAKDMFKEFPSSSKYFLKKDGSTYKSGDLFVQKDLAKTLKLIAKEGPDAFYKGKIADLIAADMAKNKGIITKEDLAQYQAIWRKPVEGTYRGYDIISMSPPSSGGAHIIEILNIMENANIENLGFASSKTLHIMAEAMRQAYADRSEYMGDPDFVKIPLDKLTSKEYAKEIYAKIPKDKALPSSKVKPGLGQIHEGHNTTHYSVLDSKGNAVSITYTINASYGSGATVEGAGFLLNDEMDDFSIKPGVPNLYGLVGGEANAIEPKKRPLSSMSPTIILKDGKVFMVVGSPGGSRIITTVLQVISNVIDHKMDISTAVESPRFHMQWLPDEIRTEPFGIIKDVQNNLEKMGYKITKEPYMGDVNAIMVDPKTGKIIGSMDTRKEF

SEQ ID NO: 22 - DNA, 20 bp, Artificial
  Synthetic oligonucleotide
atgggatttcgtattaacac

SEQ ID NO: 23 - DNA, 24 bp, Artificial
  Synthetic oligonucleotide
ctgtagtaatcttaaaacattttg

(19) United States
(12) Patent Application Publication
(10) Pub. No.: US 2007/0249553 A1
(43) Pub. Date: Oct. 25, 2007

(54) Vaccine And Nucleic Acids Capable Of Protecting Poultry Against Colonisation By Campylobacter

(75) Inventors: Diane Newell, Addlestone (GB); Shaun Cawthraw, Addlestone (GB)

Correspondence Address: THE MCCALLUM LAW FIRM, P. C., 685 BRIGGS STREET, PO BOX 929, ERIE, CO, 80516, US

(73) Assignee: The Secretary Of State For Environment, Food & Rural Affairs

(21) Appl. No.: 11/666,477

(22) PCT Filed: Oct. 25, 2005
(86) PCT No.: PCT/GB05/04102

§ 371 Date: Jul. 2, 2007

(30) Foreign Application Priority Data
Oct. 26, 2004 (GB) 0423681.6

(51) Int. Cl. A61K 31/7052; A61P 43/00; C07H 21/00; C12N 15/12

(52) U.S. Cl. 514/44; 435/320.1; 536/23.1

(57) ABSTRACT

Nucleic acids encoding Campylobacter proteins, in particular antigenic proteins such as a flagellin, or encoding a variant thereof, or a fragment of either of these, are capable of protecting poultry such as chickens, against colonisation by Campylobacter, and so may be used in veterinary therapy or prophylaxis. This has an impact on human health.

The present invention relates to vaccines and nucleic acids capable of protecting poultry against colonisation by Campylobacter, in particular Campylobacter jejuni, as well as to veterinary compositions containing these and their preparation. The invention further comprises foodstuffs obtained as a result of this treatment, and its use in the prevention of infection by Campylobacter of the human population. Campylobacter spp., principally Campylobacter jejuni and Campylobacter coli, are major human intestinal pathogens. C. jejuni is the major cause of foodborne disease in the UK causing over 40,000 reported cases per annum. Campylobacter infection has an incubation period of between 2-10 days. Symptoms include high fever, abdominal pain, and profuse diarrhoea.

Identified vehicles of infection include contaminated drinking and recreational waters, raw cows' milk and undercooked poultry meat. Campylobacter spp. can be isolated with high frequency from poultry and products derived from them, from cattle and a variety of wild animals. They are also widely present in the natural environment. Epidemiological studies indicate that the handling and consumption of poultry meat is a major risk factor. Up to 95% of UK broiler flocks are asymptomatically colonised with this organism and on-farm control or prevention of flock colonisation is a priority for regulatory authorities.

However, attempts to prevent flocks becoming colonised using biosecurity methods have been generally unsuccessful (Newell & Fearnley, 2003, Appl. Environ. Microbiol. 69: 4343-4351). Therefore an effective animal vaccine, in particular one that is effective in poultry, would be desirable.

It has been found that chickens colonised with live campylobacters generate both circulating and mucosal specific antibody responses (Cawthraw et al., 1994, Avian Dis. 38: 341-9). The major antigen against which these antibodies have been induced appears to be flagellin.

The term “flagellin” refers to a bacterial protein, which arranges itself in a hollow cylinder to form the filament in bacterial flagellum. These proteins are generally named in accordance with the order in which the genes encoding them appear in the genome of the organism. Thus Flagellin A (or “Fla A”) is encoded by flaA, the first flagellin gene in the genome. FlaA is found upstream of the flaB gene encoding Flagellin B. Flagellin A tends to be expressed in much higher amounts. However, the sequences of flaA and flaB are highly homologous, and they can crossover.

Preliminary studies indicate that antibody responses generated with a live infection are partially protective (Cawthraw et al., 2003, Int. J. Med. Microbiol. 293 Suppl. 35: 30). Clearly however, in this case, the concept of using whole bacterial cells as live vaccines would be unacceptable, as it would potentially exacerbate the problem.

However, several attempts to generate protective responses in poultry using killed antigens or subunit vaccines have been generally unsuccessful. For instance, administration of vaccine preparations including the flagellin antigen to poultry has been found to produce an antibody response, but this response was not protective against colonisation.

The concept of using DNA as a vaccine was initially described in 1990 (Wolf et al., 1990, Science 247: 1465-1468). In that paper it was demonstrated that direct intramuscular injection of purified bacterial plasmid DNA in mice resulted in the expression of an encoded reporter gene. The use of DNA vaccines for immunising poultry against certain diseases has also been described (Oshop et al., 2002, Vet. Immunol. Immunopathol. 89: 1-12). A number of potential DNA vaccines were tested with varying degrees of success. Some of these vaccines appear to provide at least partial protection whilst others appear to have no effect.

According to the present invention there is provided a nucleic acid encoding a Campylobacter protein, or encoding a variant thereof, or a fragment of either of these, which nucleic acid is capable of protecting poultry against colonisation by Campylobacter, for use in veterinary therapy or prophylaxis.

Hitherto there has been no suggestion that a DNA vaccine could reduce colonisation by Campylobacter.

However the applicants of the present invention have found that administration of a DNA vaccine can protect species such as poultry against colonisation by Campylobacter species and in particular by Campylobacter jejuni. The protection appears to be independent of detectable antibody response.

The expression “variant” as used herein refers to sequences of amino acids which differ from the or a base sequence from which they are derived or compared in that one or more amino acids within the sequence are substituted for other amino acids, but which retain the ability of the base sequence to protect poultry against infection and/or colonisation by Campylobacter. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide. Suitably variants will be at least 60% identical, preferably at least 70% identical, more preferably at least 75% identical, and yet more preferably at least 90% identical to the base sequence.

Identity in this instance can be judged for example using the BLAST program or the algorithm of Lipman-Pearson, with Ktuple:2, gap penalty:4, Gap Length Penalty:12, standard PAM scoring matrix (Lipman & Pearson, 1985, Science 227: 1435-1441).

The term “fragment thereof” refers to any portion of the given amino acid sequence which has the same activity as the complete amino acid sequence and/or which has the ability to protect poultry against infection and/or colonisation by Campylobacter. Fragments will suitably comprise at least 5 and preferably at least 10 consecutive amino acids from the basic sequence.

Suitably, the nucleic acid encodes an antigenic Campylobacter protein thereof or a variant thereof or a fragment of either. Examples of such proteins include flagellin, peptidyl-prolyl cis-trans isomerase, outer membrane fibronectin-binding protein, a protein of a multidrug efflux system (cmeA, B or C), a chaperonin, a periplasmic protein, an elongation factor TU, thioredoxin, a major outer membrane protein, a CiaB protein, an enzyme such as phospholipase A, gamma-glutamyl transpeptidase as well as some hypothetical proteins.

Particular examples of such proteins are:

FlaA illustrated by SEQ ID NO: 1,

FlaB illustrated by SEQ ID NO: 3,

Peb 4 illustrated by SEQ ID NO: 4,

Peb 3 illustrated by SEQ ID NO: 5,

Peb 2 illustrated by SEQ ID NO: 6,

Peb 1 illustrated by SEQ ID NO: 7,

CadF illustrated by SEQ ID NO: 8,

Cme A, B & C illustrated by SEQ ID NOs 9, 10 and 11 respectively,

GroEL (cpn60) illustrated by SEQ ID NO: 12,

GroES (cpn10) illustrated by SEQ ID NO: 13,

Cj0420 (putative periplasmic protein) illustrated by SEQ ID NO: 14,

Tuf (Cj0470) illustrated by SEQ ID NO: 15,

TrxA illustrated by SEQ ID NO: 16,

PorA—major outer membrane protein illustrated by SEQ ID NO: 17,

CiaB illustrated by SEQ ID NO: 18,

PldA illustrated by SEQ ID NO: 19,

Cj0447 (hypothetical protein) illustrated by SEQ ID NO: 20, or

Ggt illustrated by SEQ ID NO: 21.

The above-referenced sequences are provided in the listing of sequences below. Sequences for the genes encoding these Campylobacter jejuni proteins are available in the literature and/or in Genbank.

In particular, the protein is a flagellin.

The nucleotide sequences of Campylobacter flagellin genes can vary considerably. A short variable region (SVR) between positions 450 and 500 is flanked by regions of conserved sequences. Fragments will suitably be derived from these conserved regions.

Preferably the nucleic acid encodes a Campylobacter flagellin sequence.

The flagellin may be one which is obtainable from any Campylobacter species which colonises poultry such as C. jejuni, C. coli, or Campylobacter lari. In particular however, the flagellin is one which is obtainable from C. jejuni or C. coli, and most preferably from C. jejuni.

Suitable Campylobacter flagellins include FlaA or FlaB. In particular the nucleic acid encodes Campylobacter Flagellin A or a variant thereof, or a fragment of any of these.

A particularly preferred nucleic acid encodes Flagellin A obtainable from Campylobacter jejuni strain NCTC 11168, the amino acid sequence of which is provided in SEQ ID NO: 1. The wild-type nucleic acid encoding Flagellin A of Campylobacter jejuni strain NCTC 11168 is shown in SEQ ID NO: 2.

Other flagellin amino acid sequences, and the corresponding gene coding sequences, are shown in Nuitejen et al., 1992, Campylobacter jejuni, Current Status and Future Trends, Nachamkin et al. (Eds), American Society for Microbiology, Washington D.C., USA, pp 282-296; Meinersman et al. 1997, J. Clin. Microbiol. 35: 2810-2814; and Meinersman & Hiett, 2000, Microbiol. 146: 2283-2290.

Suitable nucleic acids include SEQ ID NO: 2 or modifications thereof.

As used herein, the term “modification” used in relation to a nucleic acid sequence means any substitution of, variation of, modification of, replacement of, deletion of, or the addition of one or more nucleic acid(s) from or to a polynucleotide sequence providing the resultant protein sequence encoded by the polynucleotide exhibits the same properties (for example, antigenic properties) as the protein encoded by the basic sequence. The term therefore includes allellic variants and also includes a polynucleotide which substantially hybridises to the polynucleotide sequence of the present invention. Preferably, such hybridisation occurs at, or between low and high stringency conditions. In general terms, low stringency conditions can be defined as 3×SSC at about ambient temperature to about 55° C. and high stringency condition as 0.1×SSC at about 65° C. SSC is a buffer containing 0.15 M NaCl and 0.015 M tri-sodium citrate (pH 7.0). 3×SSC is three times as strong as SSC and so on.

Typically, modifications have 62% or more of the nucleotides in common with the polynucleotide sequence of the present invention, more typically 65%, preferably 70%, even more preferably 80% or 85% and, especially preferred are 90%, 95%, 98% or 99% or more identity.

When comparing nucleic acid sequences for the purposes of determining the degree of identity, programs such as BESTFIT and GAP (both from Wisconsin Genetics Computer Group (GCG) software package). BESTFIT, for example, compares two sequences and produces an optimal alignment of the most similar segments. GAP enables sequences to be aligned along their whole length and finds the optimal alignment by inserting spaces in either sequence as appropriate. Suitably, in the context of the present invention when discussing identity of nucleic acid sequences, the comparison is made by alignment of the sequences along their whole length.

Particular examples of nucleic acids include nucleic acids which encode amino acid sequences of any one of SEQ ID NOs 1 or 3-21. A particular example of a nucleic acid which encodes SEQ ID NO: 1 is SEQ ID NO: 2.

The nucleic acid is suitably administered to poultry species for protection against colonisation by Campylobacter. Poultry in this instance includes chickens, turkeys and game birds such as ducks, quails etc. In particular, the poultry species is a chicken.

As used herein the expression “capable of protecting poultry against colonisation” means that the poultry is less susceptible to colonisation by the organism. This may be achieved by preventing each individual bird within a flock from becoming colonised (so there is no “first bird” which is then responsible for transmission to the remainder of the flock). However, once Campylobacter has colonised a flock, efficacy is achieved by reducing colonisation levels as compared to a flock which is not vaccinated, in particular by 2×log reduction, which brings the colonisation levels below that which would cause a risk to human health.

The nucleic acids of this invention are particularly suitable for use as “naked DNA” or “naked RNA” vaccines. Consequently they are for example suitably incorporated into plasmids that express in vivo in host cells.

Particular examples of plasmids suitable for use in the present invention include the pCMV-link plasmid, which is publicly available.

Thus a further aspect the invention provides a plasmid which includes a nucleic acid encoding a Campylobacter protein, or encoding a variant of a Campylobacter protein, or encoding a fragment of either of these, wherein the nucleic acid is capable of protecting poultry against colonisation by Campylobacter, for use in veterinary therapy.

The plasmid described is suitably mixed with a pharmaceutically acceptable carrier for administration as a vaccine. Therefore a third aspect of the invention provides a veterinary composition comprising a pharmaceutically acceptable carrier and a plasmid including the nucleic acid sequence as described above, in combination with a veterinarily acceptable carrier.

In an alternative aspect, the invention provides a veterinary composition comprising a pharmaceutically acceptable carrier and a nucleic acid sequence as described above, in combination with a veterinarily acceptable carrier.

Also provided according to the present invention is a vaccine comprising a nucleic acid encoding a Campylobacter protein, or encoding a variant of a Campylobacter protein, or encoding a fragment of either of these, wherein the nucleic acid is capable of protecting poultry against colonisation by Campylobacter. The vaccine may alternatively or additionally comprise the plasmid as described herein. The vaccine may additionally comprise a pharmaceutically acceptable carrier and/or a veterinarily acceptable carrier. The vaccine is particularly suited for use in veterinary therapy.

The vaccine is preferably acellular, i.e. contains no live or killed whole cell components.

The vaccine or formulation comprising DNA and/or RNA (including plasmids comprising DNA and/or RNA) may be injected into poultry whose own cellular machinery translates the nucleic acid into the Campylobacter protein, or variant thereof, or fragment of either of these. The protein, variant or fragment may be presented in the context of MHC class I molecules, and therefore be capable of inducing a brisk cellular immune response in contrast with traditional vaccines which produce mainly a humoral immune response. The nucleic acid of the vaccine may be transferred into the host cell by retrovirus, vaccinia virus or adenovirus vectors or by attachment to cationically charged molecules such as liposomes, calcium salts or dendrimers. Alternatively, the desired nucleic acid may be directly inserted into a plasmid and the naked DNA and/or RNA injected. Naked plasmid DNA vaccines bypass the problem of safety and manufacturing issues arising when viral vectors are used, and also avoid complications or interference from an immune response directed at a delivery vector.

The pharmaceutically acceptable carrier in compositions or vaccines of the invention may be liquid or solid. The compositions of the invention may be formulated for parenteral administration and in particular intramuscular injection, although other means of application are possible as described in the pharmaceutical literature, for example administration using a Gene Gun. Orally delivered formulations are preferred and in ovo or topical formulations are also suitable.

Formulations or vaccines may include also adjuvants, and in particular plasmid adjuvants such as CpGs, DNA encoding cytokines such as Interleukins, CaPO₄ or adjuvantising lipids, such as lipofectin.

The dosages used will vary depending upon the animal being treated, the age and size of the animal, and its disease status. These factors will be determined using conventional clinical practice. Generally speaking however, for administration to poultry as a prophylactic, dosage units of from 0.25 μg to 1 mg may be employed.

Booster doses may be given if desired or necessary. In particular, the applicants have found that a dosage regime in which the nucleic acid is given at least twice over a period of time before exposure to Campylobacter is extremely effective at providing protection.

According to a further aspect of the invention, there is provided a method of protecting poultry against colonisation by Campylobacter, which method comprises, administering to the poultry, a nucleic acid, expression vector or vaccine as described above.

According to another aspect of the invention, there is provided the use of a nucleic acid or expression vector as described above in the preparation of a vaccine for use in the prophylaxis or therapy of Campylobacter colonisation.

By treating the poultry population in this way, it is possible to protect from infection by Campylobacter the human population who consume the poultry.

Thus in yet a further aspect of the invention, there is provided a method of protecting a human from Campylobacter infection, the method comprising administering to the poultry population in the food chain, a nucleic acid, a plasmid, or a composition as described herein.

Alternatively the invention provides the use of a nucleic acid, a plasmid, or a composition as described above in the protection of humans against infection by Campylobacter, by reducing colonisation in the poultry population of the food chain.

In yet a further aspect, the invention provides a foodstuff comprising poultry which has, before slaughter, been treated with a nucleic acid, a plasmid, or a composition as described herein.

All references cited herein are incorporated by reference in their entirety.

In order that the invention may be more fully understood, a preferred embodiment of DNA vaccine, in accordance therewith, will now be described by way of example only and with reference to the accompanying diagrammatic drawings in which:

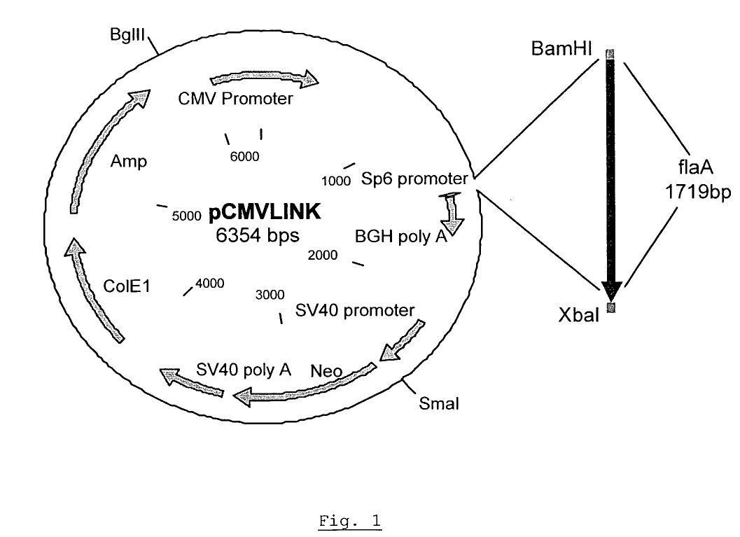

FIG. 1 is a map showing construction of plasmid pCMV-CjflaA used in the preparation of a DNA vaccine in accordance with the invention; and

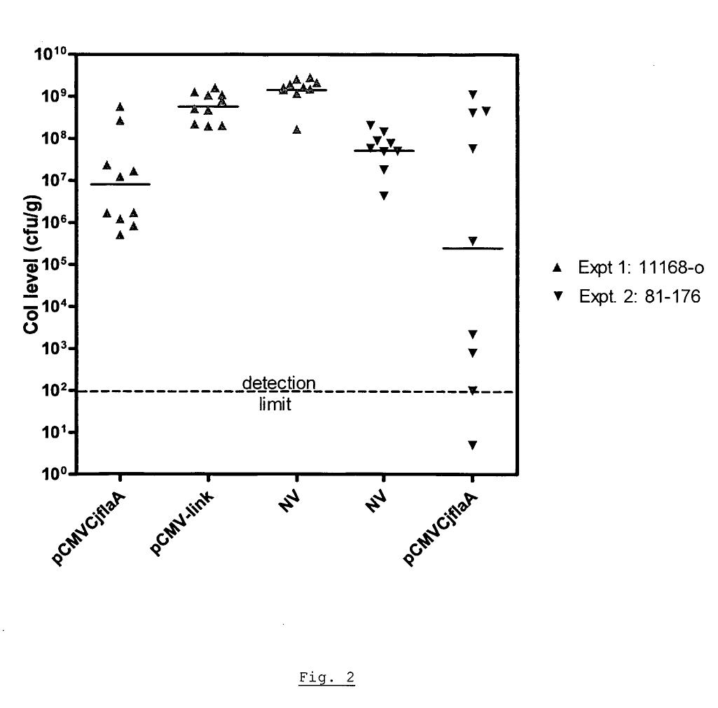

FIG. 2 shows caecal colonisation levels (cfu/g) of birds immunised with a plasmid DNA vaccine with or without the flagellin gene (pCMV-link and pCMVCjflaA respectively), and of birds that were not vaccinated (NV).

### EXPERIMENTAL

Bacterial Strains

Campylobacter jejuni strain 11168-O (Gaynor et al., 2004, J. Bacteriol. 186: 503-17) was used as the source of bacterial DNA and as a challenge strain. Campylobacter jejuni strain 81-176 was used as a heterologous strain for a challenge study. Bacteria were grown overnight at 42° C. on agar plates containing 10% defibrinated sheep blood in an atmosphere of 8% O₂, 7% CO₂, and 85% N₂. One Shot® TOPO F′ E. coli cells (Invitrogen) were used as the recipient in cloning reactions. Transformants were selected on Luria-Bertani (LB) agar containing ampicillin (100 μg/ml).

Construction and Preparation of DNA Vaccines

The construction of the control plasmid pCMV-link has been described previously (Chambers et al., 2000, Clin. Infect. Dis. 30 Suppl. 3: S283-287). The plasmid is based on pcDNA3.1 from Invitrogen (Leek, the Netherlands). The plasmid pCMV-CjflaA was constructed by inserting the flagellin. (flaA) gene of C. jejuni strain 11168-O into the multiple cloning region of pCMV-link. The flaA gene was amplified by PCR from C. jejuni strain 11168-O as a 1719 bp product using the following primers: forward: 5′-ATG GGA TTT CGT ATT AAC AC-3′ (SEQ ID NO: 22) and reverse: 5′-CTG TAG TAA TCT TAA AAC ATT TTG-3′ (SEQ ID NO: 23) (Wassenaar & Newell, 2000, Appl. Environ. Microbiol. 66: 1-9). The flaA PCR amplicon was ligated into the pCR®2.1-TOPO® vector (Invitrogen) using the TOPO®/PCR cloning kit and electroporated into One Shot® TOPO F′ E. coli cells (Invitrogen). The pCR®2.1-TOPO® flaA plasmid was then purified (Qiagen) and digested with restriction enzymes BamHI and XbaI to give a 1812 bp product containing the 1719 bp flaA gene. This product was then ligated into the BamHI and XbaI sites of pCMV-link (FIG. 1) to give a 8049 bp plasmid pCMV-CjflaA. The plasmid was electroporated into One Shot® TOPO F′ E. coli cells (Invitrogen).

Plasmid DNA for immunisation was prepared using a QIAGEN-tip 10000 plasmid extraction kit with endotoxin-free buffers (Qiagen, Crawley, UK), following manufacturer's instructions.

Vaccination

In the first experiment, three groups of 10 specific, pathogen-free (SPF) chicks (Lohmann's, Germany) were housed in separate isolators. At 2 days of age, birds were immunised as follows: group 1—no treatment, group 2—71 μg pCMV-link DNA in 100 μl PBS, intramuscularly in the thigh, group 3—71 μg pCMV-CjflaA DNA in 100 μl PBS, intramuscularly in the thigh. Similar inoculations were given when the birds were 18 days of age. At 25 days of age, all birds were dosed by oral gavage with 2.2×10³ cfu C. jejuni strain 11168-O in 0.1 ml PBS.

Previous studies have demonstrated that with an oral dose of 3×10³ cfu strain 1168O, maximal colonisation (approximately 10⁹ cfu per gram caecal contents is achieved in chickens within 5 days of challenge (Gaynor et al., 2004, supra). Therefore, birds were killed 5 days later and caecal colonisation levels determined by the culturing of serial dilutions on selective media as described previously (Wassenaar et al., 1993, J. Gen. Microbiol. 139: 1171-1175).

In the second experiment a group of SPF chicks (n=9) was vaccinated as above but with 57.8 μg pCMV-CjflaA DNA at 4 and 18 days of age. A second group (n=9) was untreated. At 25 days of age, all birds were dosed by oral gavage with 1.87×10³ cfu C. jejuni strain 81-176 in 0.1 ml PBS. Birds were killed 5 days later and caecal colonisation levels determined as before.

Results

### Experiment 1

Groups of birds, vaccinated with pCMV-link DNA, pCMV-CjflaA DNA or untreated, were challenged with 2.2×10³ cfu C. jejuni strain 11168-O, and caecal colonisation levels were determined 5 days later. The results are shown in Table 1 below and FIG. 2. In FIG. 2 the individual colonisation levels are given as the cfu per gram of caecal contents. The bar equals the geometric mean level. The results clearly indicate that DNA vaccination can reduce the levels of colonisation by about 2×log₁₀. Birds vaccinated with pCMV-CjflaA DNA were colonised significantly less than those given pCMV-link (p=0.007) and the untreated (p<0.0001).

An ELISA technique (Cawthraw et al., 1994, Avian Dis. 38: 341-349) was used to detect circulating and mucosal antibodies directed against C. jejuni flagellin. No specific antibodies were detected in any of the groups. Thus protection appears to be independent of detectable antibody responses.

TABLE 1

| | | No. | Geom. | |
|---|---|---|---|---|
| Group | Treatment | Colonies | mean | Range |
| 1 | — | 10/10 | 1.4 × 10⁹ | 1.7 × 10⁸-2.9 × 10⁹ |
| 2 | pCMV-link | 10/10 | 5.8 × 10⁸ | 2.0 × 10⁸-1.6 × 10⁹ |
| 3 | pCMV-CjflaA | 10/10 | 8.2 × 10⁶ | 5.2 × 10⁵-5.8 × 10⁸ |

### Experiment 2

Groups of birds, vaccinated with pCMV-CjflaA DNA or untreated, were challenged with 1.87×10³ cfu C. jejuni strain 81-176, and caecal colonisation levels were determined 5 days later. The results are shown in Table 2 and FIG. 2.

One bird from the vaccinated group had no detectable campylobacters and 3 others were colonised at low levels (<3×10³ cfu/g). The difference in the geometric means was almost significant (p=0.0503). Despite obvious protection in at least 4/9 birds, the significance was not as great as that seen in experiment 1. This is due to 3 of the birds in the vaccinated group in experiment 2 being more heavily colonised than any of the control birds. However, the colonisation levels in these birds (c. 10⁹ cfu/g) are in the normal range of maximum colonisation levels (5×10⁷-5×10⁹ cfu/g) seen for this strain in numerous other studies.

TABLE 2

| | | No. | Geom. | |
|---|---|---|---|---|
| Group | Treatment | colonies | mean | Range |
| 1 | — | 9/9 | 5.2 × 10⁷ | 4.4 × 10⁶-2.1 × 10⁸ |
| 2 | pCMV-CjflaA | 8/9 | 2.1 × 10⁵ | 0-1.1 × 10⁹ |

These results indicate that effective protection against Campylobacter colonisation can be achieved using DNA vaccines based upon flagellin.

Although the present invention has been described with reference to preferred or exemplary embodiments, those skilled in the art will recognize that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

1-21. (canceled)

22. A composition comprising;
an isolated nucleic acid encoding a Campylobacter protein, a nucleic acid encoding a variant thereof, or a fragment of either of these, wherein said nucleic acid is capable of protecting poultry against colonisation by Campylobacter.

23. The composition of claim 22, wherein said Campylobacter protein is selected from SEQ ID 1, SEQ ID 3, SEQ ID 4, SEQ ID 5, SEQ ID 6, SEQ ID 7, SEQ ID 8, SEQ ID 9, SEQ ID 10, SEQ ID 11, SEQ ID 12, SEQ ID 13, SEQ ID 14, SEQ ID 15, SEQ ID 16, SEQ ID 17, SEQ ID 18, SEQ ID 19, SEQ ID 20, or SEQ ID 21

24. The composition of claim 22, wherein said nucleic acid encodes a Campylobacter flagellin sequence.

25. The composition of claim 24, wherein said Campylobacter flagellin sequence is FlaA or FlaB.

26. The composition of claim 25, wherein said Campylobacter flagellin sequence is FlaA.

27. The composition of claim 24, wherein said Campylobacter flagellin sequence is from Campylobacter jejuni or Campylobacter coli.

28. The composition of claim 27, wherein said Campylobacter flagellin sequence is from Campylobacter jejuni.

29. An isolated DNA the nucleotide sequence of which comprises SEQ ID NO. 2 or variants of SEQ ID No: 2.

30. The isolated DNA of claim 29, wherein said DNA is used for protecting poultry against colonisation by Campylobacter.

31. A plasmid comprising the nucleic acid of claim 29.

32. A veterinary composition comprising:
a plasmid according to claim 31 and a veterinarily acceptable carrier.

33. A composition comprising;
an isolated nucleic acid encoding a Campylobacter protein selected from SEQ ID 1, SEQ ID 3, SEQ ID 4, SEQ ID 5, SEQ ID 6, SEQ ID 7, SEQ ID 8, SEQ ID 9, SEQ ID 10, SEQ ID 11, SEQ ID 12, SEQ ID 13, SEQ ID 14, SEQ ID 15, SEQ ID 16, SEQ ID 17, SEQ ID 18, SEQ ID 19, SEQ ID 20, or SEQ ID 21, a nucleic acid encoding a variant thereof, or a fragment of either of these and a veterinarily acceptable carrier.

34. The composition of claim 33, further comprising a plasmid.

35. The composition of claim 33, wherein said composition is used to treat poultry and reduce colonization by Campylobacter in same.

36. A method of protecting poultry from colonisation by Campylobacter, said method comprising;
administering to the poultry an isolated nucleic acid encoding a Campylobacter protein selected from SEQ ID 1, SEQ ID 3, SEQ ID 4, SEQ ID 5, SEQ ID 6, SEQ ID 7, SEQ ID 8, SEQ ID 9, SEQ ID 10, SEQ ID 11, SEQ ID 12, SEQ ID 13, SEQ ID 14, SEQ ID 15, SEQ ID 16, SEQ ID 17, SEQ ID 18, SEQ ID 19, SEQ ID 20, or SEQ ID 21, a nucleic acid encoding a variant thereof, or a fragment of either of these.

37. The method of claim 36, wherein said poultry is chicken.

38. The method of claim 36, further comprising a veterinarily acceptable carrier.

39. A composition comprising:
poultry treated prior to slaughter according to the method of claim 15.

40. A method of protecting a human from Campylobacter infection, said method comprising;
administering to poultry an isolated nucleic acid encoding a Campylobacter protein selected from SEQ ID 1, SEQ ID 3, SEQ ID 4, SEQ ID 5, SEQ ID 6, SEQ ID 7, SEQ ID 8, SEQ ID 9, SEQ ID 10, SEQ ID 11, SEQ ID 12, SEQ ID 13, SEQ ID 14, SEQ ID 15, SEQ ID 16, SEQ ID 17, SEQ ID 18, SEQ ID 19, SEQ ID 20, or SEQ ID 21, a nucleic acid encoding a variant thereof, or a fragment of either of these and a veterinarily acceptable carrier.

41. The method of claim 40, wherein said human is prevented from becoming infected by Campylobacter due to a decreased colonisation of said Campylobacter in the poultry population.
